(19) United States
(12) Patent Application Publication
(10) Pub. No.: US 2008/0065021 A1
(43) Pub. Date: Mar. 13, 2008

(54) Surgical instrument

(75) Inventors: Andrew E. Jenkins, Rhondda Cynon Taff (GB); Kester J. Batchelor, Minneapolis, MN (US)

Correspondence Address: NIXON & VANDERHYE, PC, 901 NORTH GLEBE ROAD, 11TH FLOOR, ARLINGTON, VA, 22203, US

(73) Assignee: Gyrus Medical Limited, St. Mellons (GB)

(21) Appl. No.: 11/523,807

(22) Filed: Sep. 20, 2006

(30) Foreign Application Priority Data
Sep. 7, 2006 (GB) 0617599.6

(51) Int. Cl. A61M 5/178

(52) U.S. Cl. 604/167.02

(57) ABSTRACT

A surgical system comprises a tubular member (8), a surgical instrument (1) and a sealing assembly (23). The surgical instrument (1) is locatable within the tubular member (8), the tubular member having a distal end locatable at a surgical operation site which is supplied with pressurised gas, and a proximal end remote from the surgical operation site. The surgical instrument (1) has a distal end locatable at the surgical operation site. The sealing assembly (23) is provided at the proximal end of the tubular member (8) to seal the tubular member with respect to the proximal end portion of the surgical instrument (1) to prevent gas escaping from the surgical operation site through the proximal end of the tubular member. The sealing assembly (23) is constituted by a diaphragm (26) whose peripheral edge portions (26a) are fixed to the tubular member (8). The central portion (26b) of the diaphragm (26) is formed with a slit (27) through which the surgical instrument (1) passes. The sealing assembly (23) is such that movement of the surgical instrument (1) relative to the tubular member (8) causes the diaphragm (26) to move between first and second operating positions in which the slit (27) faces respectively towards and away from the distal end of the tubular member, and such that the edges of the slit sealingly engage with the surgical instrument in the first and second operating positions, and in all positions therebetween.

### BACKGROUND OF THE INVENTION

This invention relates to a surgical instrument, and in particular to a bipolar electrosurgical instrument for use in the bulk removal of tissue, as in a laparoscopic hysterectomy. The invention also relates to a surgical instrument provided with a sealing assembly for preventing the escape, through the instrument, of an inert gas which is pumped to an operation site associated with the surgical instrument.

In a laparoscopic hysterectomy, the body of the uterus is resected from the stump or fundus, and then removed from the operative site. To enable the uterus to be removed through a limited surgical opening, it is desirable to morcellate it into relatively smaller pieces of tissue, which are easier to remove. The present invention relates to an instrument and method for morcellating and removing a uterus.

U.S. Pat. Nos. 5,957,884, 6,007,512 and 6,036,681 describe examples of morcellating devices in which an element carrying an electrode is rotated in order to cause the morcellation of tissue. This rotation of the electrode necessitates a mechanical drive arrangement, which increases the complexity of the instrument.

The specification of International patent application PCT/GB2005/001922 seeks to provide a simpler, and hence more reliable, arrangement for the bulk removal of tissue, and provides a combination of a device for morcellating tissue within a body cavity of a patient and a tissue-pulling device. The morcellating device comprises a stationary tube having a distal end portion, the tissue-pulling device being locatable within the tube. The combination includes a bipolar electrosurgical electrode assembly including first and second electrodes, the first electrode being located at the distal end of the tube, such that, when an electrosurgical cutting voltage is applied to the electrode assembly, the tissue-pulling device can be moved to pull tissue against the distal end of the tube to form a core of severed tissue within the tube, and further moved in order to remove the severed tissue from the body cavity of the patient.

The morcellating device of this known system operates under an inert gas such as CO₂, with the current flow from the active electrode to the return electrode being via tissue. The inert gas is introduced to the operation site either through the morcellating instrument itself, or via a separate endoscopic instrument. A disadvantage of the known instrument is that the inert gas can pass from the operation site, through the stationary tube to exit from the proximal end of the tube where the tissue-pulling device passes through that end of the tube. Escape of the inert gas is particularly of concern when the tissue-pulling device is operated, as the relative movement between the tissue-pulling device and the stationary tube facilitates the escape of inert gas from the interior of the tube. The escape of inert gas is undesirable, particularly where it leads to surges in the flow of inert gas supplied to the surgical site.

It is known to provide a seal at the proximal end of the tube to reduce the escape of inert gas, and typically this is constituted by a duckbill valve. The disadvantage of such a valve is that, as morcellated tissue is withdrawn through the tube, it tends to engage the edges of the duckbill valve, thereby at least partially opening the valve, and hence leading to an undesirable escape of inert gas. The engagement of morcellated tissue with such a valve also impedes proximal movement of the tissue, and risks tearing of the valve and/or loss of grip of the tissue.

An aim of the invention is to provide an improved seal for the proximal end of a morcellating surgical instrument.

### SUMMARY OF THE INVENTION

The present invention provides a surgical system comprising a housing, a surgical instrument and a sealing assembly, the surgical instrument being locatable within the housing, the system being usable for surgery in which a surgical operation site is supplied with pressurised gas, the housing having a proximal end locatable outside the body of a patient, the surgical instrument having a distal end locatable at the surgical operation site, and the sealing assembly being provided at the proximal end of the housing to seal the housing with respect to the proximal end portion of the surgical instrument to prevent gas escaping from the surgical operation site through the proximal end of the housing, wherein the sealing assembly is constituted by a diaphragm whose peripheral edge portions are fixed to the housing, the central portion of the diaphragm being formed with a slit through which the surgical instrument passes, the sealing assembly being such that movement of the surgical instrument relative to the housing causes the diaphragm to move between first and second operating positions in which the slit faces respectively towards and away from the proximal end of the housing, and such that the edges of the slit sealingly engage with the surgical instrument in the first and second operating positions, and in all positions therebetween.

When the seal is in its second operating position, it faces the proximal end of the surgical instrument, thereby offering a proximally-tapering configuration to the surgical instrument (or to tissue associated with the surgical instrument), which minimises impediment to proximal movement and reduces the chance of the seal tearing.

Preferably, a tubular member constitutes the housing, the tubular member having a distal end locatable at the surgical operation site.

In a preferred embodiment, the system further comprises an extension piece at the proximal end of the tubular member, the proximal end of the extension piece having a circular cross-section, and the diaphragm being fixed with its circumferential edge portion within the circumferential proximal end portion of the extension piece. The extension piece may be fixed to the proximal end of the tubular member.

Preferably, the cross-sectional area of the proximal end of the extension piece is larger than the proximal end portion of the tubular member.

The tubular member may have a circular cross-section.

Preferably, the central portion of the diaphragm is substantially dome-shaped, and is connected to the circumferential edge portion thereof by a thin annular section.

Advantageously, the slit in the central portion is longitudinally offset from the circumferential edge portion of the diaphragm when the sealing assembly is in its first and second operating positions, and the sealing assembly is such that the dome-shaped central portion faces respectively towards and away from the proximal end of the tubular member when the diaphragm is in its first and second operating positions.

In a preferred embodiment, the central portion of the diaphragm is constituted by a relatively thin laminar member.

Advantageously, the thin laminar member is positioned centrally within a relatively thick circumferential edge portion of the diaphragm.

Preferably, the thin laminar member is connected to the circumferential edge portion by an annular section that is thinner than the laminar member. This thinner annular section prevents any location forces being transferred to the diaphragm edges during construction, and constitutes a deformation absorption area, which preferentially allows the seal to invert.

Advantageously, the annular connecting section is connected to an outer circumferential edge portion of the laminar member that has a thickness greater than the central portion of the laminar member, thereby providing structural rigidity to react against abdominal pressure when no device is inserted, and to encourage close mating of the thinner region with an inserted surgical instrument.

Preferably, the slit is symmetrically positioned with respect to the centre of the diaphragm, and the slit is a cross-shaped slit. The advantage of this is that, if the surgical instrument is not positioned centrally within the tubular member, it will tend to engage with only one of the triangular flaps defined by the cross-shaped slit, thereby limiting the loss of sealing capacity provided by the diaphragm.

The provision of this laminar member being a cross-shaped slit ensures that, if a tissue particle adheres to the exterior of the surgical instrument one or more of the triangular flaps defined by the slit can closely engage around such a particle, thereby maintaining the sealing action.

The sealing assembly may further comprise an annular seal positioned within the housing proximally of the diaphragm and engageable with the surgical instrument.

Advantageously, the annular seal is positioned at the proximal end of the extension piece.

In a preferred embodiment, the surgical instrument is constituted by a tissue-pulling device.

In this case, the system further comprises a morcellating device positioned at the distal end of the tubular member.

Preferably, the morcellating device is constituted by a bipolar electrosurgical electrode assembly including first and second electrodes, the first electrode being located at the distal end of the tubular member such that, when an electrosurgical cutting voltage is applied to the electrode assembly, the tissue-pulling device can be moved to pull tissue against the distal end of the tubular member to form a core of severed tissue within the tubular member, and can be further moved in order to remove severed tissue from the surgical operation site.

The surgical instrument may be constituted by an insufflation tube or a trocar.

The invention also provides a surgical port assembly comprising a housing and a sealing assembly, the assembly being usable for surgery in which a surgical operation site is supplied with pressurised gas, the housing having a proximal end locatable outside the body of a patient, the sealing assembly being provided at the proximal end of the housing to seal the housing to prevent gas escaping from the surgical operation site through the proximal end of the housing, wherein the sealing assembly is constituted by a diaphragm whose peripheral edge portions are fixed to the housing, the central portion of the diaphragm being formed with a slit through which a surgical instrument can pass, the sealing assembly being such that movement of the surgical instrument relative to the housing causes the diaphragm to move between first and second operating positions in which the slit faces respectively towards and away from the proximal end of the housing, and such that the edges of the slit sealingly engage, in use, with the surgical instrument in the first and second operating positions, and in all positions therebetween.

### BRIEF DESCRIPTION OF THE DRAWINGS

The invention will now be described in more detail, by way of example, with reference to the drawings, in which;

FIG. 1 is a schematic side view of a morcellating system constructed in accordance with the invention;

FIG. 2 is a schematic sectional view of a part of the electrosurgical instrument of the system of FIG. 1;

FIG. 3 is a view similar to that of FIG. 2, illustrating a first operational position of a seal of the electrosurgical instrument;

FIG. 4 is a view similar to that of FIG. 3, illustrating a second operational position of the seal;

FIG. 5 is a perspective view illustrating the action of the seal;

FIG. 6 is a perspective view of the seal;

FIG. 7 is a sectional view of the seal; and

FIG. 8 is a sectional view of a modified form of seal;

### DESCRIPTION OF PREFERRED EMBODIMENTS

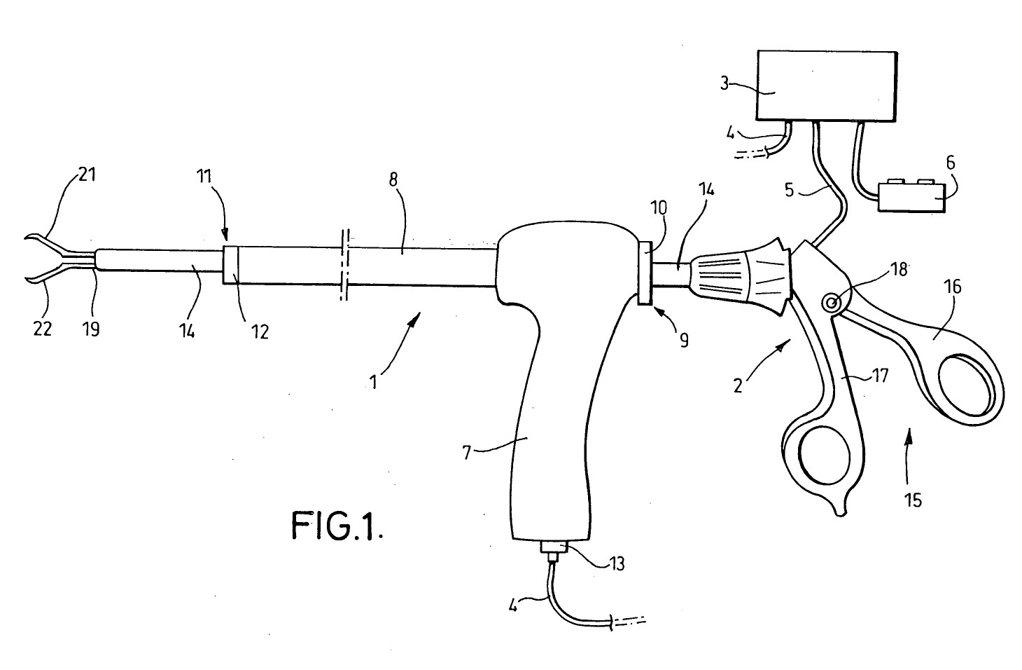

Referring to the drawings, FIG. 1 shows a morcellating system comprising a morcellating device 1, a tissue-pulling device 2, and an electrosurgical generator 3. The generator 3 is connected to the morcellating device 1 by means of a cable 4, and to the tissue-pulling device 2 by means of a cable 5. The generator 3 is controlled by means of a footswitch 6.

The morcellating device 1 comprises a handle 7 and a cylindrical tube 8. The cylindrical tube 8 is hollow, and defines a lumen therein. The proximal end 9 of the tube 8 extends from the handle 7 as shown at 10, and the distal end II of the tube is provided with an electrosurgical electrode assembly 12. The electrosurgical electrode assembly 12 comprises an active tissue-cutting electrode (not shown), and an insulating member (not shown), both extending around the circumference of the tube 8. The insulating member separates the active electrode from the remainder of the tube 8, which acts as a return electrode.

The tube 8 is connected to one pole of the generator 3, via the cable 4 and a connector 13. The active electrode extends around the entire circumference of the tube 8, and is connected to the other pole of the generator 3, via the cable 4, the connector 13 and additional wiring (not shown). In this way, the active and return electrodes constitute the bipolar electrode assembly 12, which, when energised by the generator 3, is capable of cutting tissue coming into contact with the distal end 11 of the tube 8.

The tissue-pulling device 2 comprises a tubular shaft 14, at the proximal end of which is a scissors-type handle mechanism 15, having a first handle 16 and a second handle 17. The second handle 17 is pivotable with respect to the first handle 16, about a pivot pin 18. Pivoting of the second handle 17 causes longitudinal movement of a push rod 19 extending through the shaft 14 to the distal end thereof.

A jaw assembly 20 is provided at the distal end of the shaft 14, the jaw assembly having a first jaw member 21 and a second jaw member 22 movable between open and closed positions by the movement of the push rod 19. The tissue-pulling device 2 is manually translatable in a longitudinal manner within the lumen of the morcellating device 1 by means of slidable guide members (not shown) supporting the shaft 14 of the tissue-pulling device within the tube 8. The jaw members 21 and 22 are electrically connected to the shaft 14, and the shaft is electrically connected, via the cable 5 and a connector (not shown), to the generator 3. The shaft 14 is connected to the same pole of the generator 3 as the return electrode constituted by the tube 8.

The operation of the morcellating system is as follows. The tube 8 of the morcellating device 1 is inserted into the body of a patient, either directly or through a trocar (not shown), and brought into position adjacent to the tissue to be removed (typically a resected uterus in the case of a laparoscopic hysterectomy). The tissue-pulling device 2 is then inserted through the lumen of the morcellating device 1. The handle 17 is operated to open the jaw assembly 20, and the tissue-pulling device 2 is manoeuvred so that tissue from the uterus is located between the jaw members 21 and 22. The handle 17 is then operated to close the jaw assembly 20, grasping tissue therein.

The surgeon operates the footswitch 6 to operate the generator 3 so that an electrosurgical cutting voltage is supplied between the tissue-cutting electrode and the return electrode. As mentioned previously, the push rod 19 and the jaw assembly 20 are also electrically connected to the same pole of the generator 3 as the tube 8, and so both the tube and the jaw assembly constitute the return electrode. With tissue firmly grasped in the jaw assembly 20, the device 2 is slowly withdrawn from the tube 8, pulling the tissue against the distal end of the tube and the tissue-cutting electrode. As the tissue contacts the tissue-cutting electrode, it is vaporised, allowing the device 2 to be withdrawn further into the tube 8. In this way, a cylindrical core of tissue is formed in the tube 8, the tissue being withdrawn though the proximal end 9 of the morcellating device 1 (which remains outside the body of the patient) for disposal.

The tissue-pulling device 2 can then be re-inserted into the tube 8 such that a further core of tissue can be removed from the body of the patient. By repeating this process, large quantities of tissue can be removed from the patient in a relatively short time, such that the entire uterus can be removed, if necessary, while still employing a laparoscopic approach.

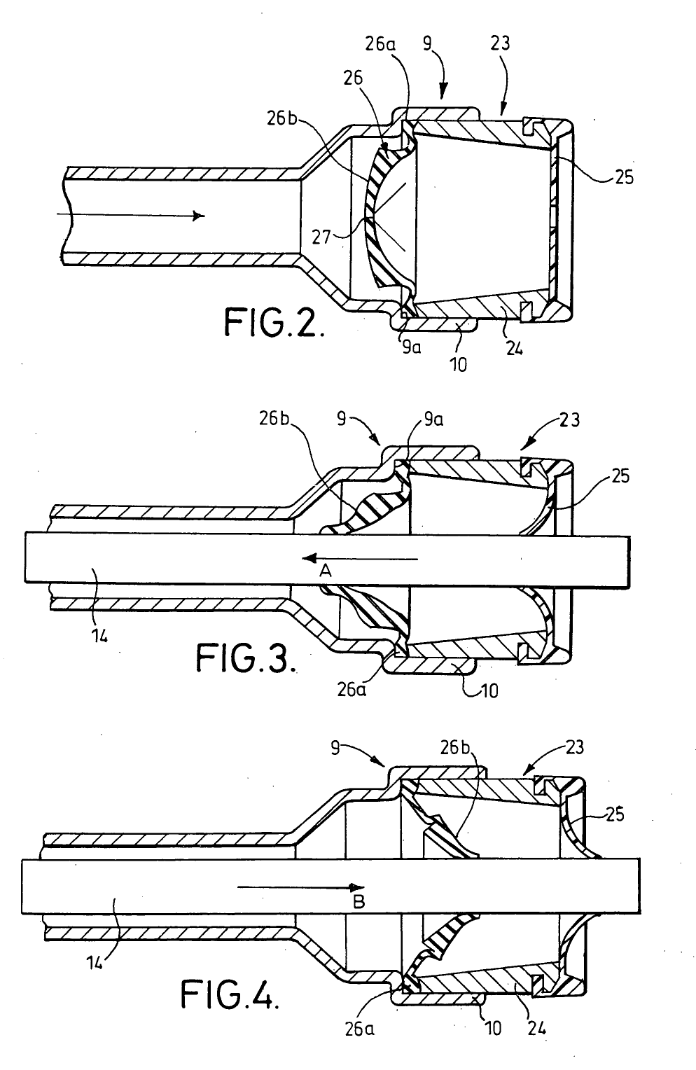

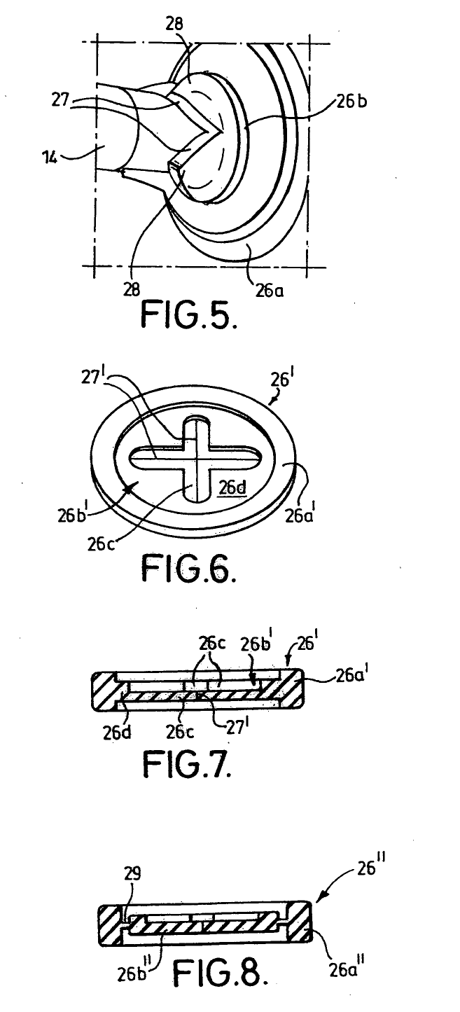

The proximal end 9 of the tube 8 is shown in greater detail in FIG. 2, this figure omitting the shaft 14 of the tissue-pulling device 2. The extension 10 of the proximal end 9 of the tube is provided with a sealing assembly 23 constituted by a generally cylindrical body 24 a rear (instrument) seal 25 and a seal 26. Both rear seal 25 and the seal 26 engage, in use, around the outer periphery of the tubular shaft 14 (see FIG. 3). The rear seal 25 has an internal diameter of 10 mm or less to complement the outer diameter of the shaft 14, and is made of a plastics material such as . . . . The seal 26 is made of a silicone rubber material, and has a circumferencial edge portion 26a and a central portion 20b, the edge portion being gripped between the cylindrical body 24 and a shoulder 9a formed in the proximal end 9 of the tube 8. The central portion 26b of the seal 26 is substantially dome-shaped, and is formed with central cross-shaped slit 27 (not shown fully in FIG. 2, but similar to the slit 27′ of the seal 26′ of FIGS. 5 and 6—to be described below).

FIG. 3 shows the seal 26 of the assembly 23 in engagement with the shaft 14 of the tissue-pulling device 2, and shows the position of the seal 26 as the tissue-pulling device moves forwardly with respect to the tube 8 so that the shaft moves in the direction of the arrow A in FIG. 3. In this position (which is shown in greater detail in FIG. 4), the four flaps 28 defined by the cross-shaped slit 27 open up and engage around the circumference of the shaft 14, thereby forming an effective gas-tight seal.

When the shaft 14 is moved in the opposite direction B (see FIG. 4), fictional engagement between the shaft and the seal 26 moves the dome-shaped seal portion 26b in such a manner as to reverse the shape of the dome so that the flaps 28 extend towards the proximal end of the tube 18, thereby forming an effective seal against the outer circumference of the shaft. Thus, respective of the position of the shaft 14 with respect to the tube 8, the seal 26 closely engages the shaft to provide an effective gas-tight seal thereagainst.

The advantage of the cross-shaped slit 27 is that an effective seal is provided even if the shaft 14 is not positioned centrally with respect to the tube 8, that is to say if the axis of the shaft is not coincident with the centre of the cross-shaped slit. Thus, in such a situation, only one of the triangular flaps 28 will tend to open as the shaft 14 moves relative to the seal 26, thereby limiting any gap between the seal and the shaft, and so limiting gas leakage.

FIGS. 5 and 6 show a modified form of seal 26′, this seal having a central portion 26b′ which is substantially in the same plane as its peripheral edge portion 26a′. The central portion 26b′, which contains a cross-shaped slit 27′ is provided in a thinner (laminar) central portion 26c of the seal 26′. This results in a more flexible sealing member which can engage more easily around tissue particles that adhere to the shaft 14, thereby improving the sealing action. The thinner central portion 26 is connected to the peripheral edge portion 26c′ by an annular portion 26d of intermediate thickness. The portions 26c and 26d meet at a right-angled shoulder 26c. Alternatively, the shoulder would be angled.

FIG. 7 shows a modification of the seal shown in FIGS. 5 and 6, this seal 26″ being substantially identical to the seal 26′, but having its central portion 26b″ connected to its peripheral edge portion 26a″ by a very thin annular strip 29. The annular strip 29 prevents any forces acting on the central portion 26b″ as a result of the movement of the shaft 14 being transferred to the peripheral edge portion 26a″.

It will be apparent that modifications could be made to the sealing assemblies described above. In particular, the cross-shaped slit could be replaced by a slit of a different configuration, for example a linear slit. Although a linear slit would not provide sealing properties as good as a cross-shaped slit, it will provide sufficient sealing capabilities, particularly if the slit is formed in a thin portion of soft flexible plastics material. It would, of course, also be possible to provide a cross-shaped slit having a different number of arms, thereby providing a different number of triangular flaps for sealing against the shaft of the tissue-pulling device. For example slits having three, five or six arms would provide adequate sealing.

The sealing system described above could also be used with other types of surgical instruments which are used at operation sites to which an inert gas is pumped, and which are disadvantaged by the escape of gas from the proximal ends thereof. For example, the seal assembly shown in FIGS. 2 and 3 could be used as an insufflation seal system. In that case, the shaft 14 would be replaced by an insufflation supply tube. In all other respects, the seal would work in exactly the same way.

Another possibility would be to use the seal system described above with a trocar, in which case the trocar would replace the shaft 14 shown in FIGS. 2 to 4. Here again, the seal would work in exactly the same way.

In either of these alternatives, the surgical instrument would be further modified by the addition of taps, ports and other such connections that are required for the correct operation of a trocar or an insufflation instrument.

We claim:

1. A surgical system comprising a housing, a surgical instrument and a sealing assembly, the surgical instrument being locatable within the housing, the system being usable for surgery in which a surgical operation site is supplied with pressurised gas, the housing having a proximal end locatable outside the body of a patient, the surgical instrument having a distal end locatable at the surgical operation site, and the sealing assembly being provided at the proximal end of the housing to seal the housing with respect to the proximal end portion of the surgical instrument to prevent gas escaping from the surgical operation site through the proximal end of the housing, wherein the sealing assembly is constituted by a diaphragm whose peripheral edge portions are fixed to the housing, the central portion of the diaphragm being formed with a slit through which the surgical instrument passes, the sealing assembly being such that movement of the surgical instrument relative to the housing causes the diaphragm to move between first and second operating positions in which the slit faces respectively towards and away from the proximal end of the housing, and such that the edges of the slit sealingly engage with the surgical instrument in the first and second operating positions, and in all positions therebetween.

2. A system as claimed in claim 1, wherein a tubular member constitutes the housing, the tubular member having a distal end locatable at the surgical operation site.

3. A system as claimed in claim 2, further comprising an extension piece at the proximal end of the tubular member, the proximal end of the extension piece having a circular cross-section, and the diaphragm being fixed with its circumferential edge portion within the circumferential proximal end portion of the extension piece.

4. A system as claimed in claim 3, wherein the extension piece is fixed to the proximal end of the tubular member.

5. A system as claimed in claim 3, wherein the cross-sectional area of the proximal end of the extension piece is larger than the proximal end portion of the tubular member.

6. A system as claimed in claim 2, wherein the tubular member has a circular cross-section.

7. A system as claimed in claim 3, wherein the central portion of the diaphragm is substantially dome-shaped.

8. A system as claimed in claim 7, wherein the central portion of the diaphragm is connected to the circumferential edge portion thereof by a thin annular section.

9. A system as claimed in claim 7, wherein the slit in the central portion is longitudinally offset from the circumferential edge portion of the diaphragm when the sealing assembly is in its first and second operating positions, and the sealing assembly is such that the dome-shaped central portion faces respectively towards and away from the proximal end of the tubular member when the diaphragm is in its first and second operating positions.

10. A system as claimed in claim 3, wherein the central portion of the diaphragm is constituted by a relatively thin laminar member.

11. A system as claimed in claim 10, wherein the thin laminar member is positioned centrally within a relatively thick circumferential edge portion of the diaphragm.

12. A system as claimed in claim 11, wherein the thin laminar member is connected to the circumferential edge portion by an annular section that is thinner than the laminar member.

13. A system as claimed in claim 12, wherein the annular connecting section is connected to an outer circumferential edge portion of the laminar member that has a thickness greater than the central portion of the laminar member.

14. A system as claimed in claim 1, wherein the slit is symmetrically positioned with respect to the centre of the diaphragm.

15. A system as claimed in claim 1, wherein the slit is a cross-shaped slit.

16. A system as claimed in claim 3, wherein the sealing assembly further comprises an annular seal positioned within the housing proximally of the diaphragm and engageable with the surgical instrument.

17. A system as claimed in claim 16, wherein the annular seal is positioned at the proximal end of the extension piece.

18. A system as claimed in claim 2, wherein the surgical instrument is constituted by a tissue-pulling device.

19. A system as claimed in claim 18, further comprising a morcellating device positioned at the distal end of the tubular member.

20. A system as claimed in claim 19, wherein the morcellating device is constituted by a bipolar electrosurgical electrode assembly including first and second electrodes, the first electrode being located at the distal end of the tubular member such that, when an electrosurgical cutting voltage is applied to the electrode assembly, the tissue-pulling device can be moved to pull tissue against the distal end of the tubular member to form a core of severed tissue within the tubular member, and can be further moved in order to remove severed tissue from the surgical operation site.

21. A system as claimed in claim 1, wherein the surgical instrument is constituted by an insufflation tube.

22. A system as claimed in claim 1, wherein the surgical instrument is constituted by a trocar.

23. A surgical port assembly comprising a housing and a sealing assembly, the assembly being usable for surgery in which a surgical operation site is supplied with pressurised gas, the housing having a proximal end locatable outside the body of a patient, the sealing assembly being provided at the proximal end of the housing to seal the housing to prevent gas escaping from the surgical operation site through the proximal end of the housing, wherein the sealing assembly is constituted by a diaphragm whose peripheral edge portions are fixed to the housing, the central portion of the diaphragm being formed with a slit through which a surgical instrument can pass, the sealing assembly being such that movement of the surgical instrument relative to the housing causes the diaphragm to move between first and second operating positions in which the slit faces respectively towards and away from the proximal end of the housing, and such that the edges of the slit sealingly engage, in use, with the surgical instrument in the first and second operating positions, and in all positions therebetween.
